(19) United States
(12) Patent Application Publication
(10) Pub. No.: US 2013/0053838 A1
(43) Pub. Date: Feb. 28, 2013

(54) INTERVENTIONAL RADIOLOGIC DEVICES AND METHODS FOR EMBEDDED FILTER REMOVAL

(71) Applicant: William T. Kuo, Redwood City, CA (US)

(72) Inventor: William T. Kuo, Redwood City, CA (US)

(21) Appl. No.: 13/591,056

(22) Filed: Aug. 21, 2012

Related U.S. Application Data
(60) Provisional application No. 61/575,521, filed on Aug. 22, 2011.

(51) Int. Cl. A61B 18/24

(52) U.S. Cl. 606/15

(57) ABSTRACT

An interventional radiologic device for removal of an embedded filter is provided. The device distinguishes a laser sheath with optical fibers for applying ablation energy to tissue. A removable introducer is movable within the lumen of the laser sheath and has at least two open channels to run wires. One or more sheath detachable modular adapters are used for providing ablation energy, force sensing and/or preventing bleeding. The distal open end of the laser sheath is preferably beveled with an angle up to 45 degrees. In one aspect, the introducer has a bulb-like tip, which fits against the distal end the laser sheath, yet can still be removed from the laser sheath via its proximal end. This device can be inserted over two wires simultaneously thereby allowing insertion over a wire loop configuration used to engage the apex of embedded IVC filters.

### CROSS-REFERENCE TO RELATED APPLICATIONS

This application claims priority from U.S. Provisional Patent Application 61/575,521 filed Aug. 22, 2011, which is incorporated herein by reference.

### FIELD OF THE INVENTION

This invention relates to surgical devices and medical procedures. In particular, the invention relates to interventional radiologic devices and methods for removing embedded filters.

### BACKGROUND OF THE INVENTION

Although standard techniques for removal of inferior vena cava (IVC) filters are successful in most cases, an estimated 20-40%, of retrievable IVC filters cannot be removed using basic methods and standard apparatus even when attempted within routine retrieval windows. Additionally, filter retrieval is not attempted in many patients with retrievable filters that have been embedded for a prolonged time and in most patients with permanent filter types, which are by definition considered irretrievable. All of these filters may intentionally or unintentionally become permanently embedded within the IVC. Several alternative retrieval methods have been described, but these have focused on tilted or tip-embedded retrievable filters. A few reports have described the risky endeavor of attempting to separate densely adherent filters from the underlying caval wall using basic apparatus, but there are no current devices specifically designed to remove embedded IVC filters. Additionally, many patients have a permanent filter type that was never designed for removal. In all patients with embedded filters, retrieval is not currently considered a feasible routine option for filter management. Consequently, these patients are subject to all the risks of prolonged IVC filter implantation, including the potential need for lifelong anticoagulation to reduce thrombotic risks related to prolonged filter implantation. The present invention provides new techniques (devices and methods) for controlled photothermal tissue ablation with an excimer laser sheath technique and related apparatus to facilitate retrieval of both permanent-type and retrievable-type embedded filters.

### SUMMARY OF THE INVENTION

An interventional radiologic device for removal of an embedded filter (e.g. an embedded inferior vena cava filter) is provided. The device distinguishes a laser sheath with a lumen between a proximal open end and a distal open end. The lumen defines an inner wall and an outer wall providing space for a plurality of optical fibers than run in between the walls from proximal to the distal end of the laser sheath. The optical fibers are used for tissue laser ablation at the distal end. The distal open end of the laser sheath is beveled with an angle up to 45 degrees, which is defined relative to a cross-section perpendicular to the laser sheath. In another embodiment, this angle is at least 20 degrees and up to 45 degrees.

The proximal open end of the laser sheath has at least one attachment mechanism for respectively at least one sheath detachable modular adapter. To apply ablation energy to the optical fibers for removing tissue near the distal open end of the laser sheath, one of these sheath detachable modular adapter is a laser device. Other sheath detachable modular adapter could be a laser sheath pressure device for measuring pressure applied to the laser sheath or one or more hemostatic valves for preventing bleeding through the proximal open end. In case of force sensing, the distal open end could have force sensors for measuring pressure applied to the laser sheath.

The laser sheath could be made of reinforced polymer sheath material, but in one aspect the laser sheath should be able to withstand up to 10 pounds of pressure without fracture.

A removable introducer is fitted to be movable within, and removable from, the lumen of the laser sheath. The introducer has at least two open channels between its proximal and distal ends to allow passage of at least two medical instruments through the channels of the introducer. In one aspect, the two channels are running parallel (or approximately parallel) to allow two wires to be run parallel at the same time. The introducer could have a tapered distal.

In one embodiment, the introducer defines a tip at its distal end and a body as the remainder part of the introducer. This tip could have a bulb-type shape. Physically, what is important is that the diameter of said proximal end of the tip is larger than the diameter of the body. In addition, the diameter of proximal end of the tip is at least larger than the diameter of the inner wall of the laser sheath or (ii) at least larger than the diameter of the outer wall of the laser sheath such that the proximal part/end of the tip fits against the distal open end of said the sheath. This bulb-type tip could also be tapered. Since the introducer with the bulb-type tip still needs to be removable from the laser sheath, in one aspect, at least part of the surface of the tip is made of a material relatively softer than the material of the laser sheath to allow bulb-like tip to be removed from the lumen, via its proximal end, of the laser sheath.

Embodiments of the invention has several advantages. For example, the device can be inserted over 2 wires simultaneously thereby allowing insertion over a wire loop configuration used to engage the apex of embedded IVC filters. The strength of the sheath material is custom tailored to withstand the forces necessary to remove an embedded IVC filter. The material is also puncture resistant to any sharp edges of the captured filter during removal through the sheath. Modular force meter attachments can be used to allow constant monitoring of tension applied to the embedded filter to allow operation within a safe window of tension during complex filter retrieval, thereby reducing the risks associated with complex filter removal. A special valve system (for example, but not limited to, a valve with an inflatable balloon-type diaphragm made of puncture resistant material) is designed to accommodate multiple device components simultaneously when used to remove the filter, while providing hemostasis. In addition, a dual-valve assembly option with containment chamber for the filter allows facile delivery of the filter with minimal blood loss. The sheath tip and construction is designed specifically for embedded filter removal using a proper angle and shape. The complete device and apparatus is specifically designed to allow safe and effective removal of embedded IVC filters.

### BRIEF DESCRIPTION OF THE DRAWINGS

FIG. 1 shows a device according to an exemplary embodiment of the invention.

FIG. 2 shows the distal end of a laser sheath with optical fibers according to an exemplary embodiment of the invention.

FIG. 3 shows a device according to an exemplary embodiment of the invention.

FIGS. 4A-D show introducers with a bulb-type tip according to an exemplary embodiment of the invention.

FIGS. 4E-F show introducers with a bulb-type tip with relatively softer material relative to the rest of the introducer and laser sheath according to an exemplary embodiment of the invention.

FIGS. 5A-F show an example of a wire-loop technique according to an exemplary embodiment of the invention. The images pertain to a patient with a 12-Fr Stainless Steel Permanent Greenfield filter placed 7.2 years ago at an outside hospital after suffering acute DVT and acute PE. The patient recovered from her VTE, but therapeutic anticoagulation was continued for underlying coronary artery disease. The patient developed bilateral lower extremity edema and CTV (not shown) revealed IVC stenosis and multiple filter leg penetrations through the caval wall. Filter removal was desired as a first step to alleviate her caval obstruction. (FIG. 5A) Inferior vena cavagram shows a tip-embedded Greenfield filter with focal caval stenosis (black arrows) associated with enlarged collateral vein filling (white arrows). (FIG. 5B) A wire-loop technique was used to untilt the filter and (FIG. 5C) rigid forceps were used to dissect the filter tip free from the caval wall. (FIG. 5E) A 16-Fr laser-tipped sheath was advanced over the wire-loop to capture the filter tip until meeting a strong point of resistance (arrow), and one session of photothermal ablation was performed along this point. (FIG. 5E) After further sheath advancement, a 2^{nd} point of resistance was encountered (arrow), and another session of photothermal ablation was performed distally. (FIG. 5F) Following ablation through all of the adherent tissues, the filter was completely captured within the sheath.

FIGS. 6-7 show a variation to FIG. 2 of the distal end of a laser sheath with optical fibers and pressure sensing elements according to an exemplary embodiment of the invention.

FIGS. 8A-B show a variation to the distal end of the laser sheath according to an exemplary embodiment of the invention. FIG. 8A is a view of the distal end of the laser sheath. FIG. 8B is a side-view, i.e. perpendicular to the view plane of FIG. 8A.

### DETAILED DESCRIPTION

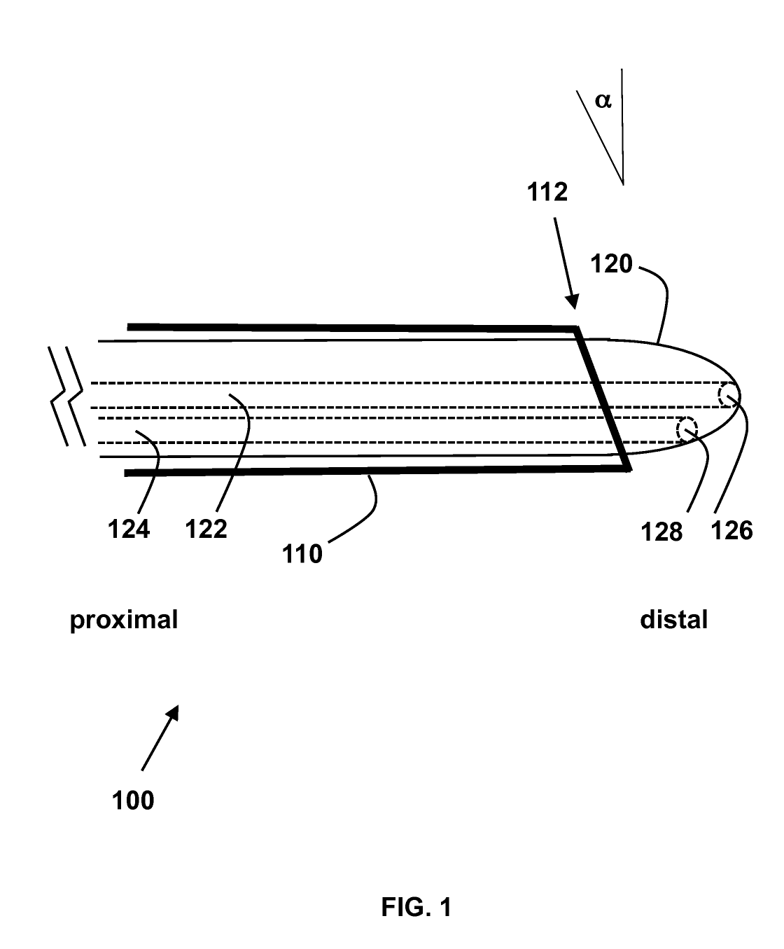

In one embodiment, the interventional radiologic device for removal of an embedded filter (for example, but not limited to, an embedded inferior vena cava filter) includes a laser sheath, one or more modular adapters, and a removable introducer. As shown by device 100 in FIG. 1, the laser sheath 110 has an inner diameter (i.e. inner wall) surrounding a lumen between a proximal open end and a distal open end. In one example, the distal open end is beveled having an angle up to 45 degrees (bevel is indicated by 112 and measures as angle α). In one example, the laser sheath is made of reinforced polymer sheath material. In another example, the laser sheath is made of reinforced polymer sheath material which is able to withstand up to 10 pounds of pressure without getting fractured. In yet another example, puncture resistant materials could be used. Through the wall of the laser sheath (220 and 230 are respectively the inner wall and outer wall of the laser sheath), optical fibers 210 are disposed between the proximal end and the distal end. A ring of laser energy provided through optical fibers 210 is capable to ablate contacted tissue around the circumference of the distal beveled tip.

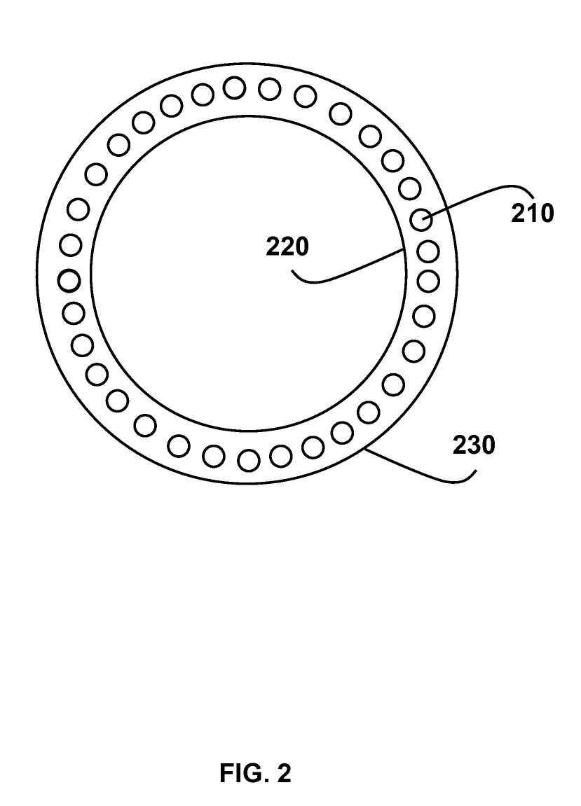

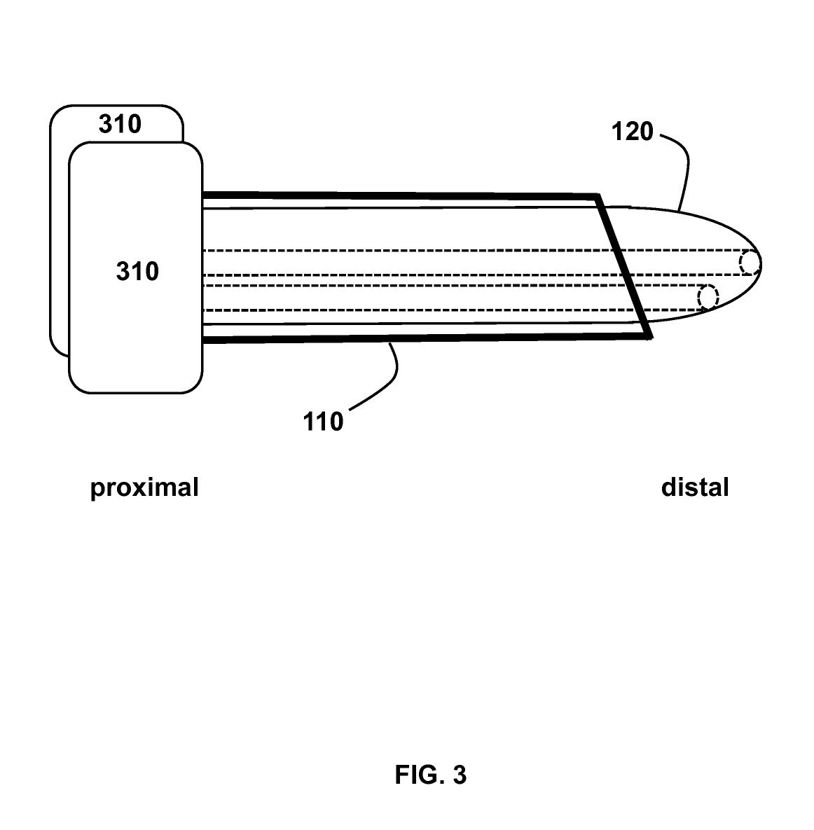

The proximal open end has at least one attachment mechanism 310 for respectively at least one sheath detachable modular adapter (FIG. 3). Examples of such adapter modules are, but not limited to, (i) a laser device for applying ablation energy to the distal open end of the laser sheath for removing tissue near the distal open end of the laser sheath (FIG. 2), (ii) a sheath pressure device for measuring pressure applied to the laser sheath, (iii) one or more special hemostatic valves for preventing bleeding through the proximal open end, and/or any combination thereof.

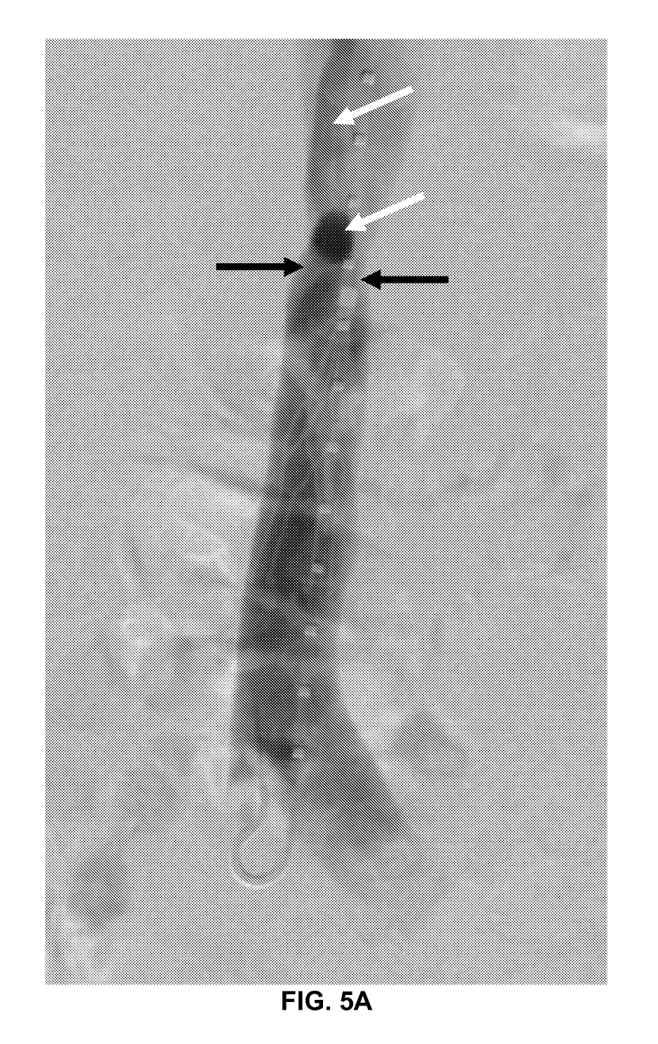

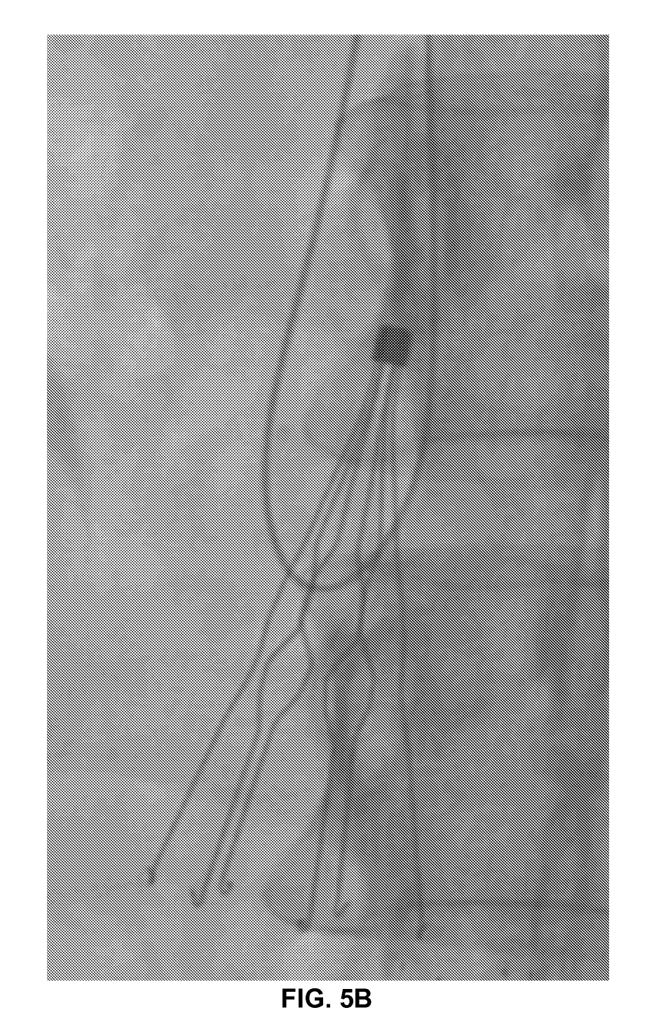

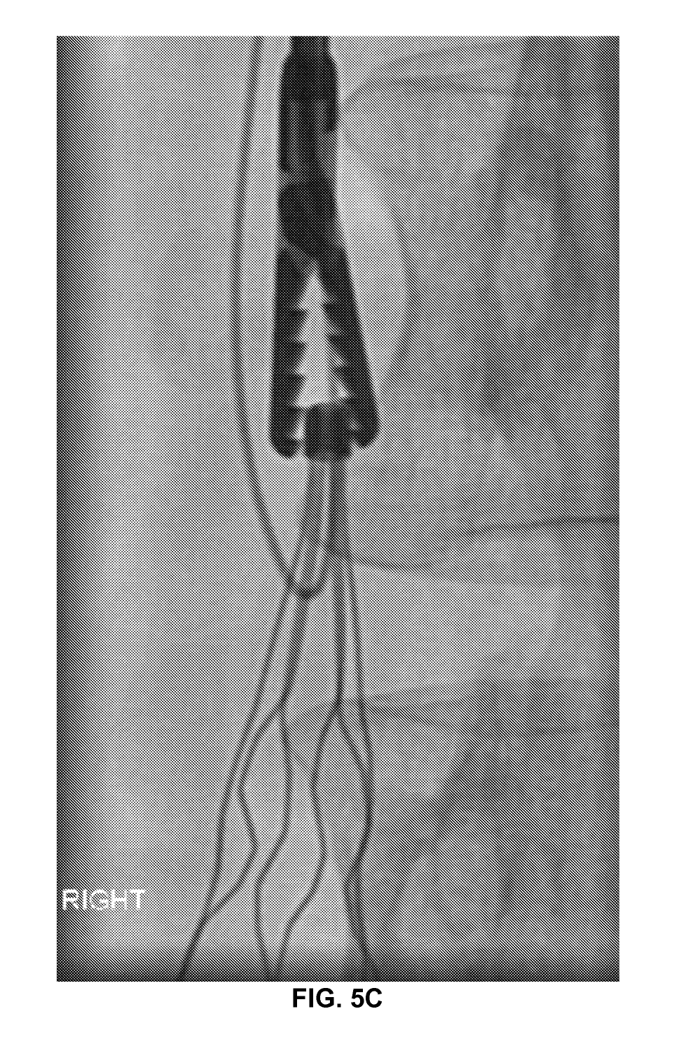

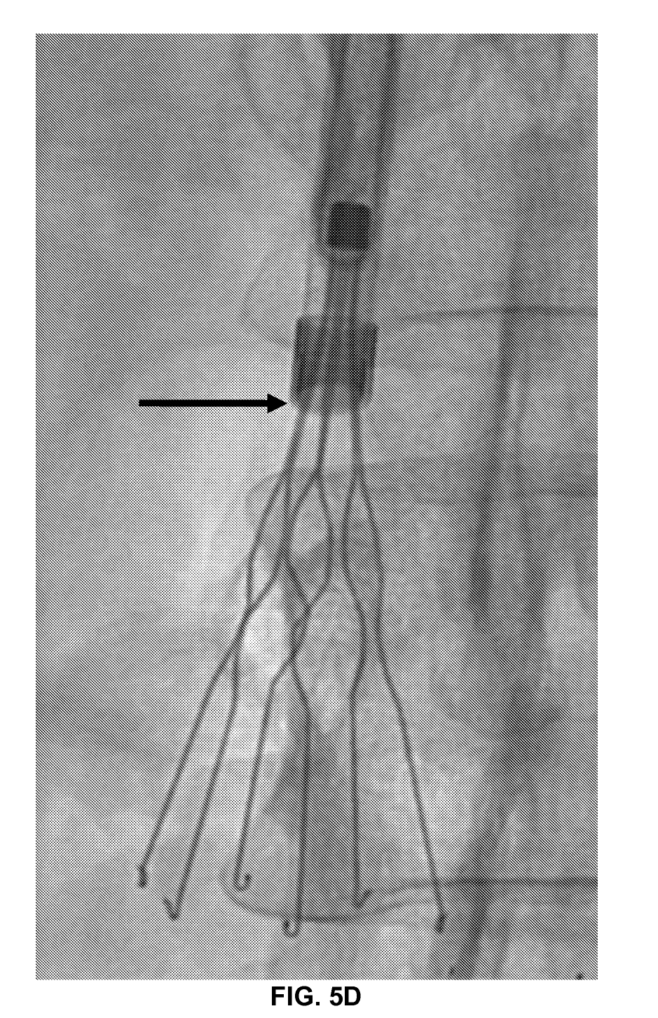

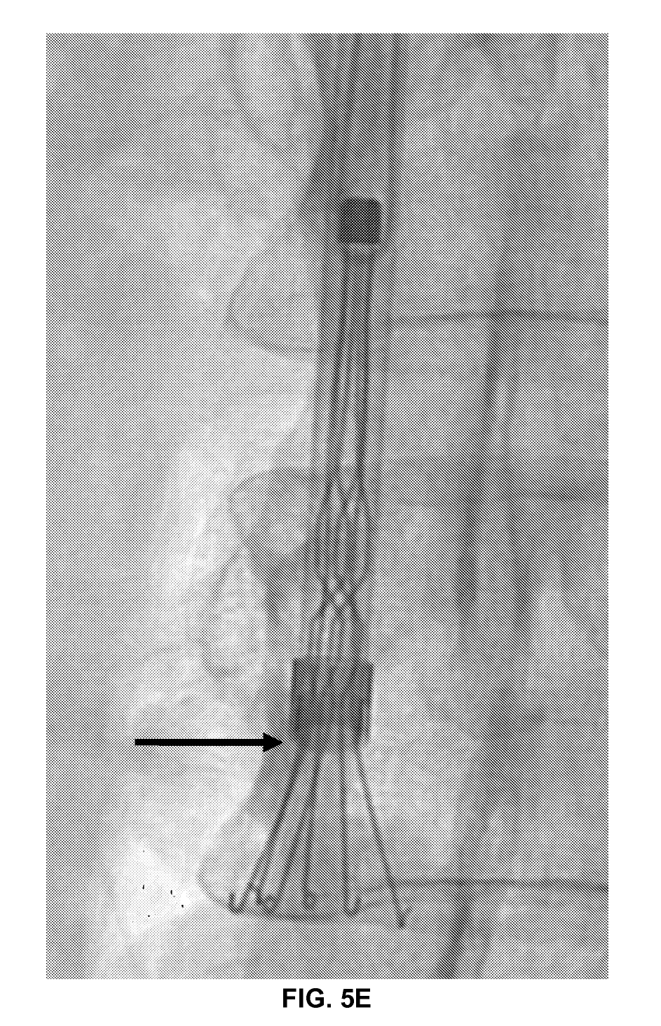

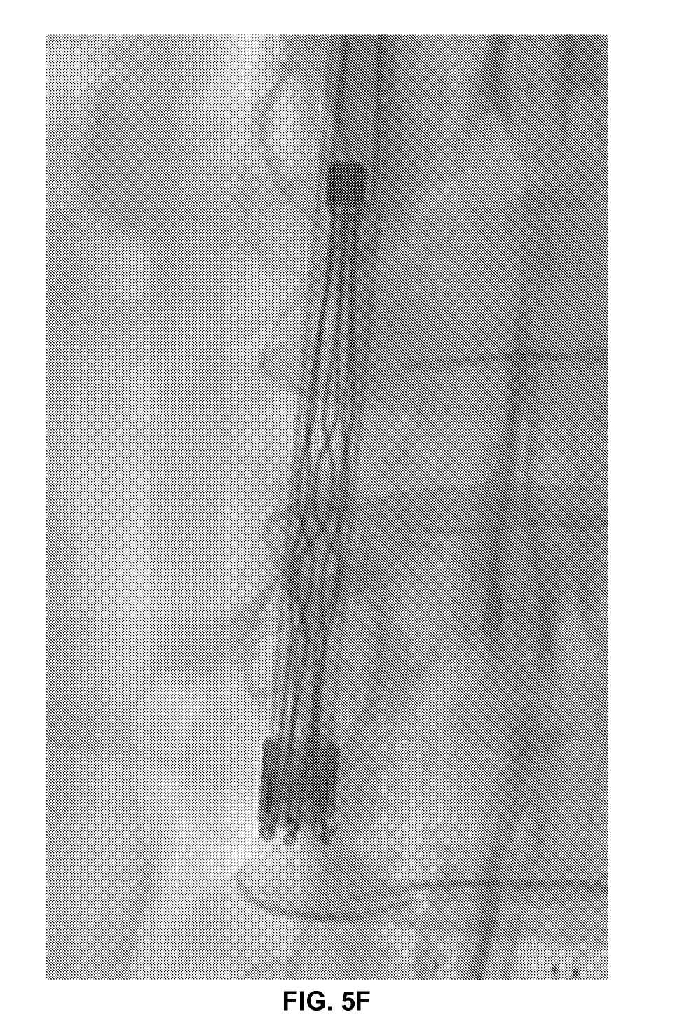

The removable introducer 120 has a proximal end and a distal end. The introducer is fitted to be movable within the inner diameter 220 of the laser sheath and is to be removable from the laser sheath when desired. The introducer further includes at least one channel 122, 124 between its proximal and distal open ends (126, 128 are open distal ends, proximal open ends not shown) for passing medical instruments. In one example, the introducer has two (or at least two) channels between its proximal and distal ends for passing medical instruments or guide wires. In another example, the introducer has two channels that are running parallel within the introducer as shown in FIG. 1. Ideally, the introducer is designed to accept two 0.035″ diameter guidewires simultaneously, which would permit placement of the laser sheath directly over a wire loop configuration (illustrated in FIGS. 5A-F).

In yet another example, the tip of the introducer is tapered as shown in FIG. 1 to fit the beveled laser sheath tip, yet still able to have two or more channels and openings at the distal end of the introducer.

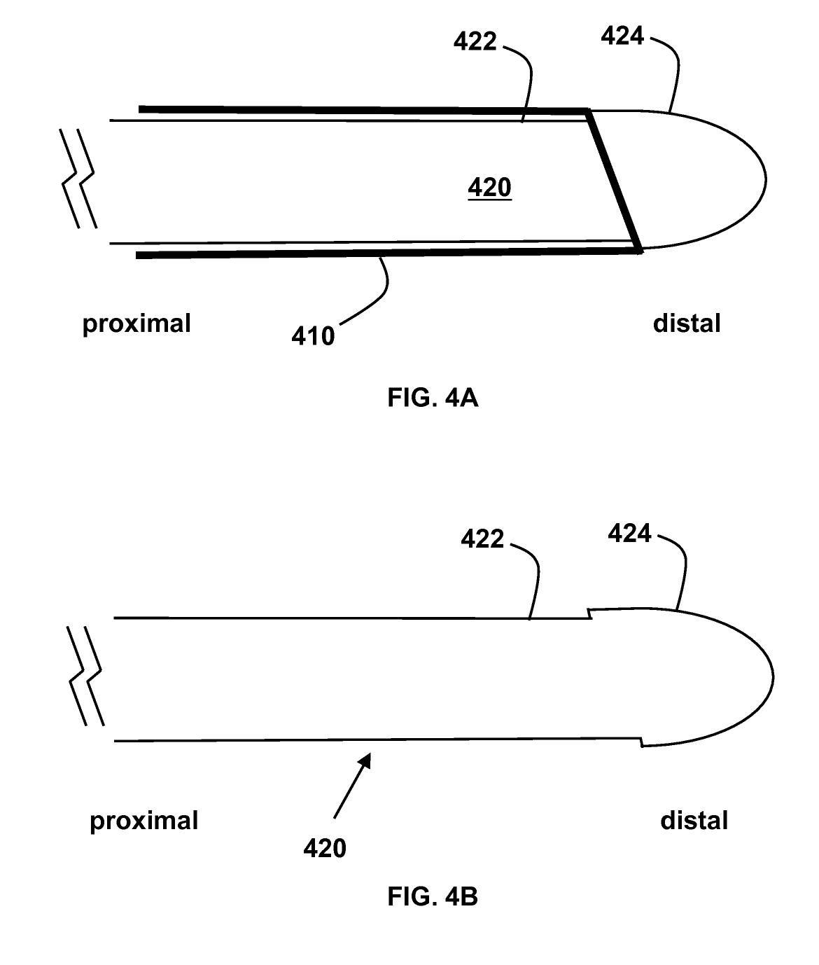

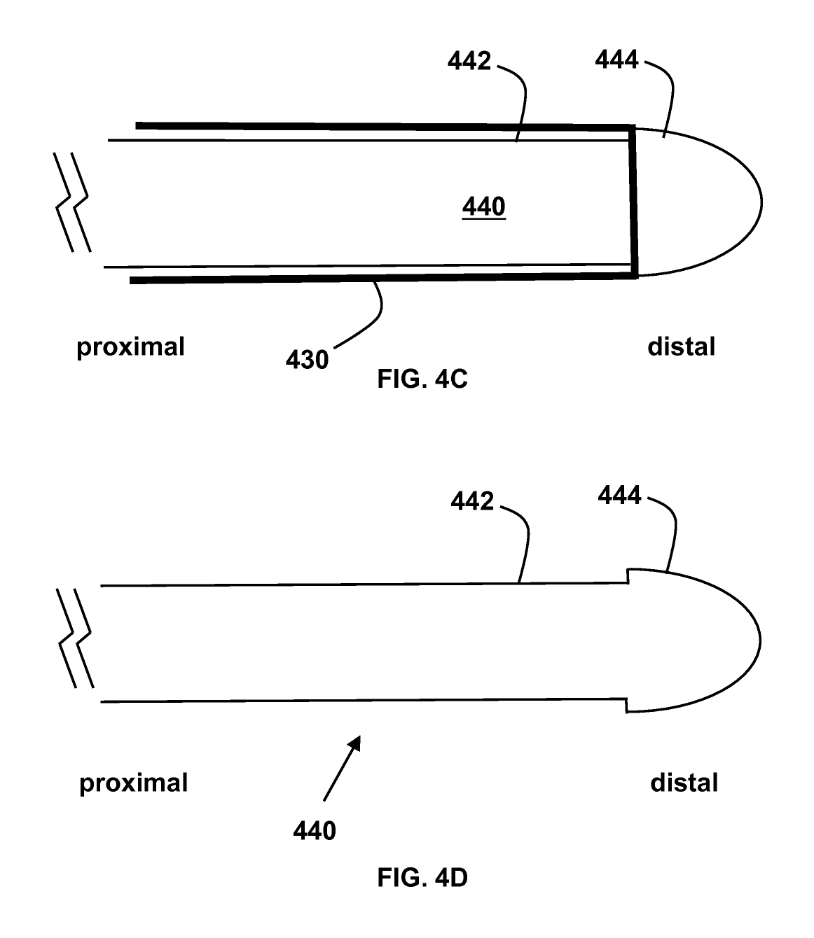

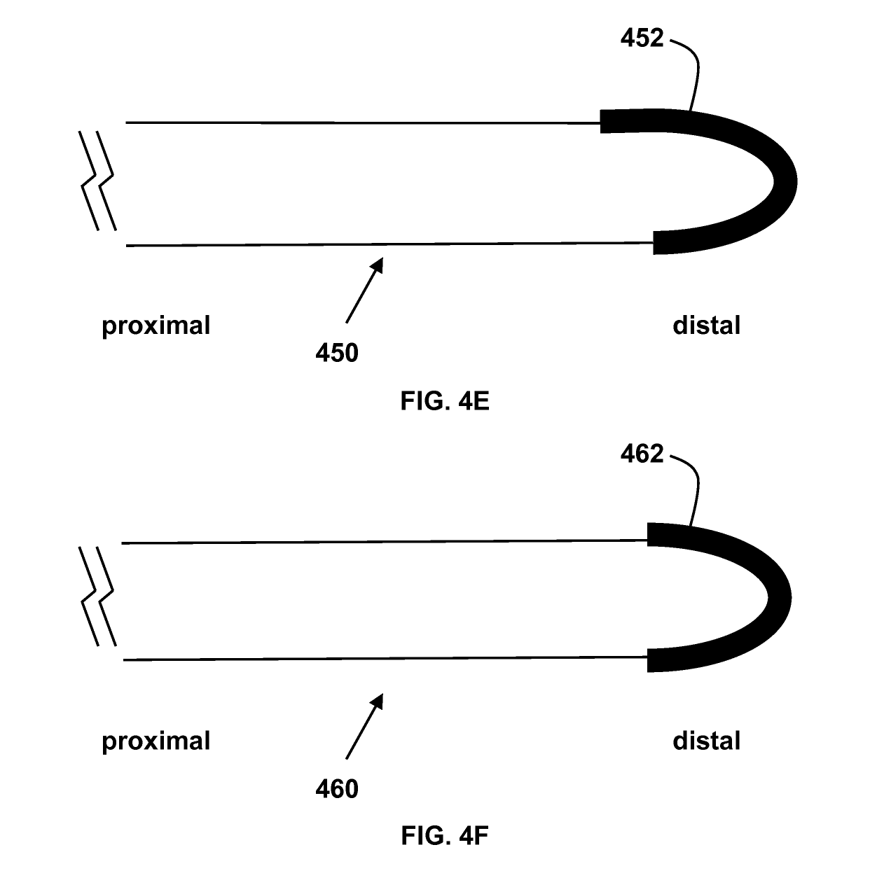

In still another example, the tip 424 of the introducer 420 confirms (at least) to the added thickness of the laser sheath 410 wall as shown in FIG. 4A. In other words, the introducer is not a cylinder that slides through the laser sheath. Instead, tip 424 of introducer 420 (note introducer has body 422 and tip 424) has become a “bulb-type” tip that is custom-shaped to fit against the distal end of laser sheath 410 and essentially fall or snap into place against the laser sheath's distal end (FIG. 4B). FIGS. 4C-D shows another embodiment of the bulb-type tip where the distal end of the laser sheath 430 is not (or virtually not) beveled. Tip 444 of introducer 442 is a bulb-type tip that fit (at least) against the distal end of laser sheath 430.

With a bulb-like shape as shown in FIGS. 4A-D, the introducer should also have a mechanism to allow release of the tip edges back through the laser sheath. In one example, the material 452, 462 around the tip (FIGS. 4E-F) of introducers 450, 460 could be softer relative to the laser sheath allowing a user to dissociate it from the laser sheath once inserted into the patient.

The following describes exemplary method steps, without any limitation, according to a procedure using the device of the invention. The filter apex must be captured using either a standard snare technique or wire loop method. If the standard snare technique is not possible, then a wire-loop must be formed around the filter apex, preferably bisecting the filter if possible. This will allow untilting of the embedded filter by placing fraction on the wire loop. If the apex is still adherent to the vessel wall, then simultaneous advancement of forceps, in parallel to the wire loop, can be used to assist tissue dissection in order to free the apex. Once free, a regular vascular sheath can then be advanced over the wire loop and filter apex to attempt filter capture. During advancement of the regular vascular sheath, if resistance is encountered, the force should be measured with a force meter attached either to the wire loop or to the sheath itself. If the amount of tension or force exceeds 6-7 pounds, there is significant scar tissue tethering the filter in place; and therefore, a laser sheath will be required to perform tissue ablation in order to free the embedded filter. If greater tension is applied without use of laser energy, there could be damage to the filter, damage to the retrieval apparatus, and/or damage to the vessel wall itself including the induction of acute thrombosis. At this point, the tension should be relinquished and the regular vascular sheath should be exchanged for a laser sheath. The laser sheath must carefully be inserted over the wire loop and then over the filter until reaching the previously identified point of resistance. The laser should then be activated to perform circumferential tissue ablation around the embedded filter components. The proper use of laser energy in this fashion will permit safe removal of the embedded filter.

Embodiments of the invention could be varied as follows. In one variation, a detachable (modular) pressure/tension gauge could be used to measure the pressure against the tissue. The gauge should be able to sustain at least 9 pounds of pressure. FIGS. 1 and 2 shown on page 2 of Appendix A in the provisional application, to which this application claims priority and which is herein incorporated by reference, is one example of such a modular pressure gauge.

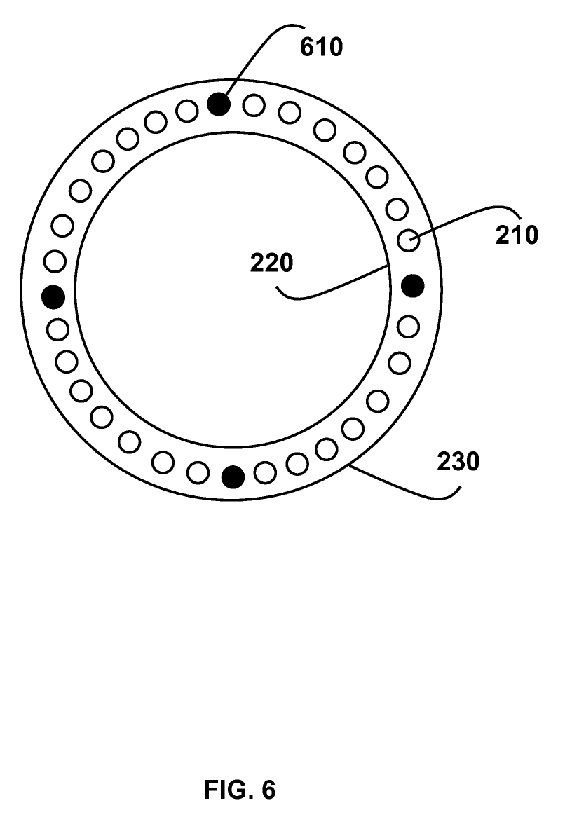

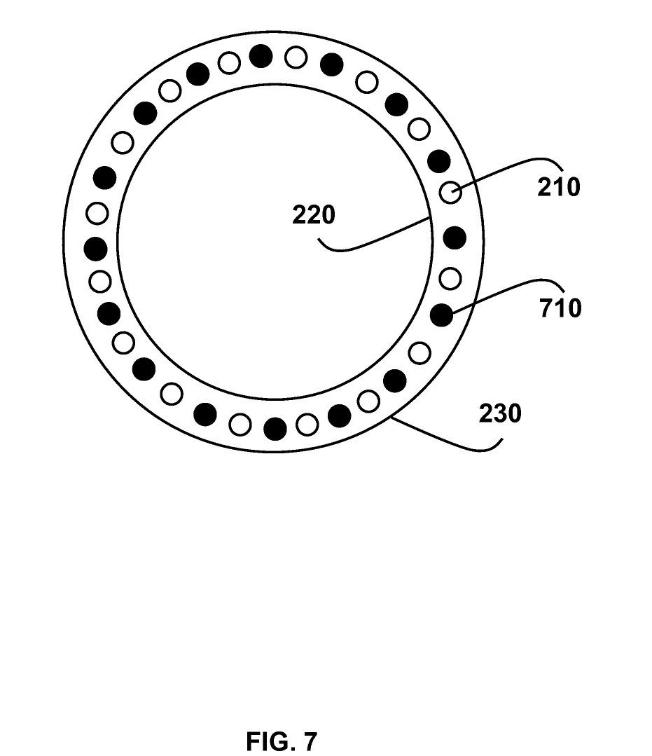

However, another example is the use of one or more pressure sensors 610, 710 at the distal tip of the laser sheath as shown in FIGS. 6-7. One could imagine either alternating force sensors with the optical fibers, having groups distributed over the tip or just one or more pressure sensors along the distal tip surface. A key aspect in design is to ensure that there are enough optical fibers to be able to perform tissue ablation as well as enough force sensors to provide pressure/force feedback to the user. Like the optical fibers being connected to a detachable unit 310 for providing ablation, the force sensors are connected to a detachable unit 310 to provide pressure sensing feedback to the user. Force sensing could also be integrated with one or more hemostatic valves. In this case, the force gauge itself could either be built into the valve itself, or alternatively, the gauge could be a separate device that could simply attach to the valve using a luer adapter.

In another variation, sheath diameters could be used such as, for example, in a range from 12-Fr up to 18 Fr to 20 Fr to accommodate filters associated with large tissue volumes.

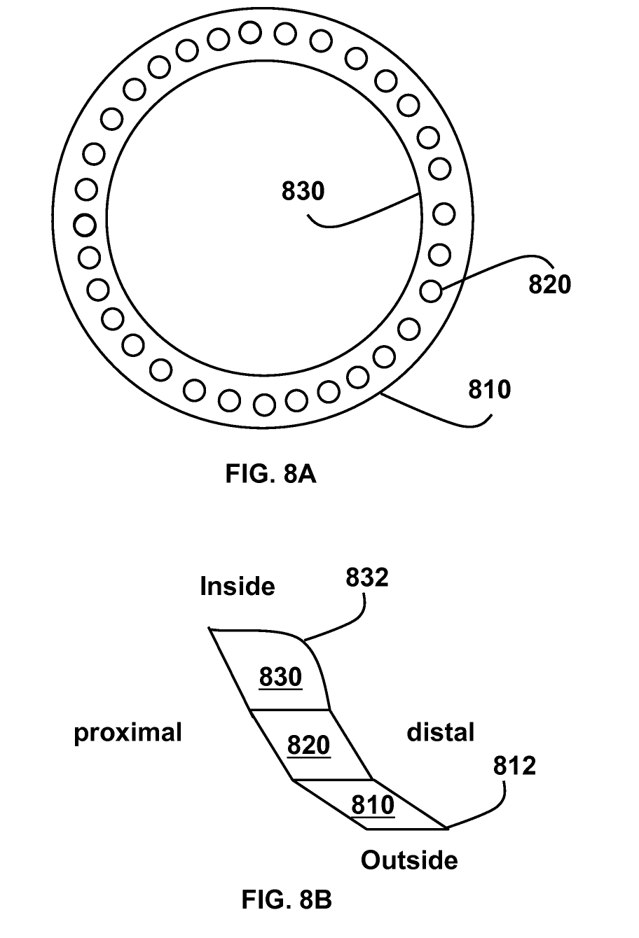

In yet another variation (FIGS. 8A-B), the outer sheath edge 810 at the distal end 812 of the laser sheath could be sharper relative to the middle layer 820 which contains the optical fiber array and relative to the inner sheath edge 830. In addition, the outer sheath edge 810 could be relatively thinner, sharper and/or metallic that the other layers to form a leading edge that can dig into the scar tissue. This digging would then help the middle sheath layer 820 to make contact against the scar tissue (not shown). The inner sheath layer 830 could also be slightly recessed at the distal tip and not sharp (i.e. relative and in view of tissue so it will not injure the guidewire coating as indicated by 832).

In still another variation, a second inflatable hemostatic valve could be added with enough space between the 2 valves to accommodate the filter as it slides out. This would allow deflation of the superficial valve to allow exit of the retrieved filter, while the deeper valve stays inflated to preserve hemostasis.

What is claimed is:

1. An interventional radiologic device for removal of an embedded filter, comprising:
(a) a laser sheath having a lumen between a proximal open end and a distal open end, wherein said lumen defines an inner wall and an outer wall, wherein said distal open end is beveled having an angle up to 45 degrees, wherein said angle is defined relative to a cross-section perpendicular to said laser sheath,
(b) a plurality of optical fibers running in between said inner and outer walls from said distal and proximal ends of said laser sheath, wherein said optical fibers at said distal end of said laser sheath are used for tissue laser ablation; and
(b) a removable introducer with a proximal end and a distal end, wherein said introducer is fitted to be movable within said lumen of said laser sheath and removable from said laser sheath, wherein said introducer comprises at least two open channels between said proximal end to said distal end to allow passage of at least two medical instruments through said introducer.

2. The interventional radiologic device as set forth in claim 1, wherein said at least two channels are running parallel within said removable introducer and wherein said medical instruments are two parallel running wires.

3. The interventional radiologic device as set forth in claim 1, wherein said removable introducer has a tapered distal end.

4. The interventional radiologic device as set forth in claim 1, wherein said introducer defines a tip at said distal end and a body as the remainder part of said introducer, wherein the diameter of proximal end of said tip is larger than the diameter of said body, and (i) wherein said diameter of proximal end of said tip is at least larger than the diameter of said inner wall of said laser sheath or (ii) at least larger than the diameter of said outer wall of said laser sheath such that the proximal end of said tip fits against said distal open end of said laser sheath.

5. The interventional radiologic device as set forth in claim 4, wherein said removable introducer has a tapered distal end.

6. The interventional radiologic device as set forth in claim 4, wherein at least part of the surface of said tip is made of a material relatively softer than the material of said laser sheath to allow said tip to be removed from said lumen of said laser sheath and through said proximal end of said laser sheath.

7. The interventional radiologic device as set forth in claim 1, wherein said proximal open end of said laser sheath comprises at least one attachment mechanism for respectively at least one sheath detachable modular adapter.

8. The interventional radiologic device as set forth in claim 7, wherein said sheath detachable modular adapter is a laser device for applying ablation energy to said plurality of optical fibers for removing tissue near said distal open end of said laser sheath.

9. The interventional radiologic device as set forth in claim 1, wherein said sheath detachable modular adapter is a laser sheath pressure device for measuring pressure applied to said laser sheath.

10. The interventional radiologic device as set forth in claim 1, wherein said sheath detachable modular adapter is one or more hemostatic valves for preventing bleeding through said proximal open end.

11. The interventional radiologic device as set forth in claim 1, wherein said embedded filter is an embedded inferior vena cava filter.

12. The interventional radiologic device as set forth in claim 1, wherein said laser sheath is made of reinforced polymer sheath material.

13. The interventional radiologic device as set forth in claim 12, wherein said reinforced polymer sheath material is able to withstand up to 10 pounds of pressure without fracture.

14. The interventional radiologic device as set forth in claim 1, wherein said distal open end comprises force sensors for measuring pressure applied to said laser sheath.

15. The interventional radiologic device as set forth in claim 1, wherein said beveled angle is at least 20 degrees and up to 45 degrees.
